(19) United States
(12) Patent Application Publication
(10) Pub. No.: US 2004/0228830 A1
(43) Pub. Date: Nov. 18, 2004

(54) Multiparticulate compositions of milnacipran for oral administration

(75) Inventors: Jane Hirsh, Wellesley, MA (US); Alison B. Fleming, Marshfield, MA (US); Roman V. Rariy, Allston, MA (US)

Correspondence Address: PATREA L. PABST, PABST PATENT GROUP LLP, 400 COLONY SQUARE, SUITE 1200, ATLANTA, GA, 30361, US

(73) Assignee: Collegium Pharmaceutical, Inc.

(21) Appl. No.: 10/766,124

(22) Filed: Jan. 28, 2004

Related U.S. Application Data
(60) Provisional application No. 60/490,060, filed on Jul. 24, 2003.
(60) Provisional application No. 60/468,470, filed on May 6, 2003.
(60) Provisional application No. 60/458,993, filed on Mar. 28, 2003.
(60) Provisional application No. 60/443,618, filed on Jan. 29, 2003.
(60) Provisional application No. 60/443,237, filed on Jan. 28, 2003.

(51) Int. Cl. A61K 31/785

(52) U.S. Cl. 424/78.12

(57) ABSTRACT

A multiparticulate milnacipran composition for oral administration has been developed. The formulation is made by complexing milnacipran with an ion-exchange resin in the form of small particles, typically less than 150 microns. Multiparticulate formulations may be any one or more of the following types of particles are formulated into a final dosage form: immediate release particles, prepared by coating drug-containing particles with the polymer that is insoluble in the neutral medium of saliva, but dissolves in the acid environment of the stomach; enteric coated particles, prepared by coating drug-containing particles with the polymer that is insoluble in the acidic environment of the stomach but dissolves in the neutral environment of the small intestines; extended release particles, prepared by coating drug-containing particles with a polymer that forms water insoluble but water permeable membrane; enteric coated-extended release particles, prepared by coating extended release drug particles with a second, enteric coating; delayed release particles, prepared by coating drug-containing particles with a polymer that is insoluble in the acidic environment of the stomach and the environment of the upper small intestines, but dissolves in the lower small intestines or upper large intestines. The various drug-containing particles described above can be further formulated into a number of different final dosage forms including, but not limited to, a liquid, liquid suspension, gel, capsule, soft gelatin capsule, tablet, chewable tablet, crushable tablet, rapidly dissolving tablet, or unit of use sachet or capsule for reconstitution.

This application claims priority under 35 U.S.C. 119 to U.S. S. No. 60/443,237 filed Jan. 28, 2003; U.S. S. No. 60/443,618 filed Jan. 29, 2003; U.S. S. No. 60/458,993 filed Mar. 28, 2003; U.S. S. No. 60/468,470 filed May 06, 2003; and U.S. S. No. 60/490,060 filed Jul. 24, 2003

### FIELD OF THE INVENTION

The present invention generally relates to novel multiparticulate milnacipran compositions for oral administration.

### BACKGROUND OF THE INVENTION

Oral formulations are available as either solid or liquid dosage forms. Solid dosage forms such as tablets or capsules are the most prevalent and convenient forms for oral administration. Typical conventional, extended, and modified release formulations of drugs are single unit dosage forms (solid tablets or coated tablets) or multiparticulate dosage forms consisting of minigranules contained in capsules (each minigranule being 0.5 millimeter in diameter or greater) that must be swallowed whole. Unfortunately such formulations are difficult to administer to patients that have difficulty swallowing or are unable to swallow due to stroke, cancer or mental impairment. In elderly patients, for example, it is common for tablets to be crushed and administered in a liquid or semi-solid vehicle. This is often the case when no liquid dosage form is available and drug needs to be administered via nasogastrointestinal or jejunostomy tube. This practice is potentially dangerous for traditional extended release formulations where, once the integrity of the tablet matrix is compromised, the entire dose of drug is “dumped”, or released immediately, leading to blood plasma levels substantially higher than the ones achieved when the formulation is properly administered. Furthermore, some sensitive patients require titration at the outset of therapy whereby the daily dose of drug is slowly increased over time until it is administered at the ultimate desired dose. For such patients a liquid formulation, where the dose administered can be easily titrated, is ideal. A liquid formulation is especially helpful when drug needs to be administered through the nasogastrointestinal or jejunostomy tube to the patients that are unconscious or completely unable to swallow.

In certain cases, however, an unpleasant taste of the drug makes conventional liquid formulation, i.e. drug dissolved in the pharmaceutically acceptable vehicle, not feasible. Milnacipran with its strong bitter taste is a perfect example of such drug. Milnacipran is a norepinephrine (NE) and serotonin (5-HT) reuptake inhibitor (NSRI) with NE to 5-HT ratio equal 2:1 (Moret et al., 1985, Neuropharmacology, 24:1211-1219; Palmier et al., 1989, Eur. J. Clin. Pharmacol., 37:235-238). Milnacipran was approved in Europe in 1996 to treat patients with depression. Its immediate release solid formulation (capsule) is available under the trade name Ixel® (Pierre Fabre). An extended release multiparticulate formulation of milnacipran comprising non-pareils coated with milnacipran was described in WO98/08495. While such a multiparticulate formulation can be sprinkled over semi-solid food and thus ameliorates some of the problems for patients with difficulty swallowing, it does not provide for convenient dose titration, taste masking, or acceptable mouth feel. It is important to note that tampering with the solid formulations (both, immediate or modified release) may result in poor dose control, leading to administration of an incorrect dose of the medicine.

What is needed is a multiparticulate formulation of milnacipran that can be formulated into any number of easy to administer and/or swallow final dosage forms including a liquid, liquid suspension, gel, capsule, soft gelatin capsule, tablet, chewable tablet, crushable tablet, rapidly dissolving tablet, or unit of use sachet or capsule for reconstitution in order to improve patient compliance and allow for convenient, flexible dose titration by the patient or the care giver.

It has been recently shown that milnacipran, in addition to being a successful antidepressant, is effective in relieving pain both associated with, and independent of, depression, such as the pain associated with chronic fatique syndrome, fibromyalgia, and in treatment of other disorders (Briley M., 2003, Curr. Opin. Investig. Drugs, 4:42-45; Cypress Bioscience Inc., Cypress Bioscience Inc. Announces Final Results of Milnacipran Phase II Clinical Trial in Fibromyalgia, Media Release, Mar. 21, 2003). See also U.S. Pat. No. 6,602,911 issued Aug. 5, 2003 and U.S. Pat. No. 6,635,675 which issued on Oct. 21, 2003.

Unfortunately, milnacipran has demonstrated numerous adverse reactions in human clinical trials with tolerability decreasing with increasing dose (Puech A. et al., 1997, Int. Clin. Psychopharm., 12:99-108). In a double-blind, randomized, multicenter clinical study, the most frequent spontaneously reported adverse events for 100 mg/day milnacipran twice daily were abdominal pain (13%), constipation (10%), and headache (9%). Interestingly, when in the same study milnacipran was given 200 mg/day twice daily, pain related adverse reactions decreased (headache to 8% and abdominal pain to 7%) but nausea and vomiting were more pronounced side effects and were reported by 7% of the patients (Guelfi J. D., 1998, Int. Clin. Psychopharm., 13:121-128). In a double-blind comparative study involving 219 elderly patients with depression, the only adverse event reported more frequently for milnacipran recipients than for TCA imipramine recipients was nausea. Patients received either milnacipran or imipramine 75-100 mg/day twice daily for 8 weeks (Tignol J. et al., 1998, Acta Psychiatrica Scandinavica, 97:157-165). It was also observed that when milnacipran was administered intravenously to 10 patients, five of them reported transient nausea. Nausea was primarily reported at the moment of peak of milnacipran plasma level (Caron J. et al., 1993, Eur. Neuropsychopharmacol., 3:493-500). This study clearly demonstrates that nausea is directly correlated with the milnacipran blood plasma concentration. In addition, it strongly suggests that the nausea can be a centrally mediated side effect since the drug was given intravenously in this study. Data from other studies suggest that milnacipran may also induce a locally mediated nausea via gastric irritation (the rapid onset of the nausea was observed even prior to achieving peak plasma levels).

The incidence of spontaneously reported milnacipran adverse experiences in placebo-controlled clinical trials is given in Table 1 (adverse effect is listed if frequency was more than 2% in milnacipran 100 mg/day group). As it can be clearly seen from data presented in Table 1, the incidence of certain adverse events increases with dosage, including nausea, vomiting, sweating, hot flashes, palpitations, tremor, anxiety, dysuria, and insomnia.

TABLE 1
Incidence of spontaneously reported milnacipran adverse
experiences in placebo-controlled clinical trials

| | Frequency of Adverse Experiences (%) | | | |
|---|---|---|---|---|
| | | 50 mg/day | 100 mg/day | 200 mg/day |
| Adverse | Placebo | twice daily | twice daily | twice daily |
| Event | N = 394 | N = 426 | N = 1871 | N = 865 |
| Nausea | 10.9 | 12.7 | 11.2 | 19.4* |
| Headache | 17.0 | 14.6 | 8.4 | 13.5 |
| Increased | 1.3 | 14.0 | 4.3* | 11.6* |
| Sweating | | | | |
| Constipation | 4.3 | 8.0 | 6.5 | 11.4* |
| Insomnia | 10.7 | 9.2 | 6.1 | 11.3 |
| Dry mouth | 5.6 | 9.4 | 7.9 | 9.0 |
| Vomiting | 3.6 | 3.8 | 3.9 | 7.9* |
| Abdominal | 5.1 | 6.1 | 6.5 | 7.6 |
| Pain | | | | |
| Tremor | 1.5 | 0.9 | 2.5 | 6.7* |
| Anxiety | 1.3 | 2.8 | 4.1 | 5.1 |
| Palpitations | 1.8 | 2.3 | 2.7 | 4.6 |
| Vertigo | 1.8 | 1.6 | 5.0 | 4.5 |
| Fatigue | 3.0 | 2.8 | 2.5 | 4.4 |
| Dysuria | 0.3 | 1.4 | 2.1* | 3.7* |
| Hot flushes | 0 | 1.6 | 3.0 | 3.6 |
| Somnolence | 3.8 | 5.4 | 2.3 | 3.5 |
| Agitation | 3.0 | 1.6 | 3.3 | 2.9 |
| Nervousness | 2.0 | 4.2 | 2.0 | 2.8 |
| Dyspepsia | 4.1 | 3.5 | 2.1 | 2.2 |

*Significantly greater than placebo

It is important to note that in one of the early depression trials, even after one week of milnacipran dose escalation employed to reduce side effects, the most commonly reported reason for discontinuation of treatment because of adverse effects was nausea and vomiting (Leinonen E., 1997, Acta Psychiatr. Scand., 96:497-504). In the recent fibromyalgia clinical trial with the long dose escalation period (four weeks) which was implemented in order to reduce milnacipran side effects and increase patient's tolerance, the most common dose-related side effect reported by patients was nausea (Cypress Bioscience Inc., Cypress Bioscience Inc. Announces Final Results of Milnacipran Phase II Clinical Trial in Fibromyalgia, Media Release, Mar. 21, 2003

The data presented in Table I demonstrates that the currently available immediate release formulation of milnacipran is not ideal for the treatment of health conditions that require milnacipran doses equal or above 100 mg/day given either once a day or twice a day due to high incidence of treatment-emergent side effects that leads to poor patient tolerance. Higher doses are required in the treatment of severe depression and other associated disorders. As shown in one of the early antidepressant clinical trials, milnacipran dosage of 200 mg/day was superior to the lower doses (Von Frenckell R et al., 1990, Int. Clin. Psychopharmacology 5:49-56). Milnacipran dosing regime of 100-250 mg daily was recently reported for the treatment of fibromyalgia (U.S. Pat. No. 6,602,911). It would be very difficult to reach the upper limits of the dose range using the currently available immediate release formulation due to the dose related treatment emergent side effects and the need to titrate over a long period to reach the required dose.

Moreover, an immediate release formulation of milnacipran may not be suitable for a once-daily dosing regimen for a treatment of depression due to milnacipran's relatively short, approximately 8 hours, half-life (Ansseau M. et al., 1994, Psychopharmacology 114:131-137). Milnacipran's half-life could also be responsible for the fact that twice-a-day administration (versus once-a-day) of immediate release formulation in fibromyalgia trial resulted in pain improvement statistically superior to that of placebo treatment (Cypress Bioscience Inc., Cypress Bioscience Inc. Announces Final Results of Milnacipran Phase II Clinical Trial in Fibromyalgia, Media Release, Mar. 21, 2003).

The ability of the patient to swallow milnacipran daily dose without tampering with the formulation becomes especially critical for modified release formulations since performance of these formulations depends on the integrity of the dosage form at the time of administration.

It is therefore the object of the present invention to provide multiparticulate milnacipran formulations which can be formulated into easy to administer and/or swallow dosage forms including, but not limited to, a liquid, liquid suspension, gel, capsule, soft gelatin capsule, tablet, chewable tablet, crushable tablet, rapidly dissolving tablet, or unit of use sachet or capsule for reconstitution.

It is a further object of the present invention to provide easy to swallow and/or administer formulations of milnacipran which are taste masked and have acceptable mouth feel.

It is still another object of the present invention to provide milnacipran multiparticulate formulations that allow for convenient, flexible dose titration to a lower or higher dose due to adjustments required for individual patient's body weight or medical necessity.

It is yet another object of the present invention to provide multiparticulate formulations of milnacipran that provide alternative pharmacokinetic release profiles with lower or reduced frequency of dosing and that eliminate or diminish unwanted side effects, especially prevalent in higher dosages.

It is another object of the present invention to provide milnacipran multiparticulate formulations that produce a therapeutic effect over approximately 24 hours when administered to a patient in need, wherein the release rate and dosage are effective to provide relief from at least one disorder selected from the group consisting of depression, fibromyalgia syndrome, chronic fatigue syndrome, pain, attention deficit/hyperactivity disorder, and visceral pain syndromes (VPS), such as irritable bowel syndrome (IBS), noncardiac chest pain (NCCP), functional dyspepsia, interstitial cystitis, essential vulvodynia, urethral syndrome, orchialgia, and affective disorders, including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, migraine headache, and tension headache, with diminished incidence and reduced intensity of common milnacipran side effects reported for immediate release formulation.

It is still another object of the present invention to provide a formulation that allows for a daily dose between 5 and 500 mg and provides for flexibility in morning or evening administration.

### SUMMARY OF THE INVENTION

A multiparticulate release milnacipran composition for oral administration has been developed. The formulation is made by complexing milnacipran with an ion-exchange resin in the form of small particles, typically less than 150 microns. To prepare a multiparticulate formulation, one or more of the following types of particles are formulated into a final dosage form:

(a) Immediate release particles, prepared by coating drug-containing particles with a polymer that is insoluble in the neutral medium of saliva, but dissolves in the acid environment of the stomach;

(b) Enteric coated particles, prepared by coating drug-containing particles with a polymer that is insoluble in the acidic environment of the stomach but dissolves in the neutral environment of the small intestines;

(c) Extended release particles, prepared by coating drug-containing particles with a polymer that forms water insoluble but water permeable membrane;

(d) Enteric coated-extended release particles, prepared by coating extended release drug particles with an enteric coating;

(e) Delayed release particles, prepared by coating drug-containing particles with a polymer that is insoluble in the acidic environment of the stomach and the environment of the upper small intestines, but dissolves in the lower small intestines or upper large intestines.

The various drug-containing particles described above can be further formulated into a number of different final dosage forms including, but not limited to, a liquid, liquid suspension, gel, capsule, soft gelatin capsule, tablet, chewable tablet, crushable tablet, rapidly dissolving tablet, or unit of use sachet or capsule for reconstitution.

A modified release multiparticulate milnacipran formulation has been developed. The modified release composition provides delayed or extended release of milnacipran to produce a therapeutic effect over approximately 24 hours when administered to a patient in need, which should result in diminished incidence and decreased intensity of common milnacipran side effects such as sleep disturbance, nausea, vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

### DETAILED DESCRIPTION OF THE INVENTION

Definitions

Modified release dosage form: A modified release dosage form is one for which the drug release characteristics of time course and/or location are chosen to accomplish therapeutic or convenience objectives not offered by conventional dosage forms such as solutions, ointments, or promptly dissolving dosage forms. Delayed release, extended release, and pulsatile release dosage forms and their combinations are types of modified release dosage forms.

Delayed release dosage form: A delayed release dosage form is one that releases a drug (or drugs) at a time other than promptly after administration.

Extended release dosage form: An extended release dosage form is one that allows at least a twofold reduction in dosing frequency as compared to that drug presented as a conventional dosage form (e.g. as a solution or prompt drug-releasing, conventional solid dosage form).

Pulsatile release dosage form: A pulsatile release dosage form is one that mimics a multiple dosing profile without repeated administration and allows at least a twofold reduction in dosing frequency as compared to the drug presented as a conventional dosage form (e.g. as a solution or prompt drug-releasing, conventional solid dosage form).

Milnacipran

Milnacipran and methods for its synthesis are described in U.S. Pat. No. 4,478,836. Milnacipran (midalcipran, midacipran, F 2207) inhibits the uptake of both norepinephrine (NE) and serotonin (5-HT), with an NE to 5-HT ratio of 2:1 (Moret et al., 1985, Neuropharmacology, 24:1211-1219; Palmier et al., 1989, Eur. J. Clin. Pharmacol., 37:235-238) but does not affect the uptake of dopamine. Milnacipran has no affinity for alpha or beta adrenergic, muscarinic, histaminergic, and dopaminergic receptors. This suggests that milnacipran has a low potential to produce anticholinergic, sedative, and stimulant effects. Milnacipran does not affect the number of beta adrenoceptors in rat cortex after chronic administration (Briley M. et al., Int. Clin. Psychopharmac., 1996, 11:10-14). Additional information regarding milnacipran may be found in the Merck Index, 12^{th} Edition, at entry 6281.

As used herein “milnacipran” also encompasses pharmaceutically acceptable, pharmacologically active derivatives of milnacipran including both individual enantiomers of milnacipran (dextrogyral and levrogyral enantiomers) and their pharmaceutically acceptable salts, mixtures of milnacipran enantiomers and their pharmaceutically acceptable salts, and active metabolites of milnacipran and their pharmaceutically acceptable salts, unless otherwise noted. It is understood that in some cases dosages of enantiomers, derivatives, and metabolites may need to be adjusted based on relative activity of the racemic mixture of milnacipran.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers. Racemates, and Resolutions”, John Wiley and Sons, Inc., 1981. Examples of resolutions include recrystallization of diastereomeric salts/derivatives or preparative chiral chromatography.

### I. Multiparticulate Milnacipran Compositions

The multiparticulate drug compositions described herein demonstrate several types of release profiles. The multiparticulate drug compositions are obtained by complexing drug with a pharmaceutically acceptable ion-exchange resin and coating such complexes.

As used herein the term “taste masking coating” refers to a pH dependent coating that is insoluble in the mouth but dissolves in the acidic pH of the stomach. As used herein the term “extended release coating” refers to a pH independent substance that will act as a barrier to control the diffusion of the drug from its core complex into the gastrointestinal fluids. As used herein, the term “enteric coating” refers to a coating material which remains substantially intact in the acid environment of the stomach, but which dissolves in the environment of the intestines. As used herein the term “delayed release coating” refers to a pH dependent coating that is insoluble in the acidic pH of the stomach, the pH within the upper small intestine, but dissolves within the lower small intestine or upper large intestine.

A. Ion-exchange Resins as Complexing Agents

Drug complexes are generally prepared by complexing the drug with a pharmaceutically acceptable ion-exchange resin. The complex is formed by reaction of a functional group of the drug with a functional group on the ion exchange resin. For milnacipran, the basic amino group can complex with an ion-exchange resin that bears an acidic group such as a sulfate or carboxylate group. Drug is released by exchanging with appropriately charged ions within the gastrointestinal tract.

Ion-exchange resins are water-insoluble, cross-linked polymers containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ioliic groups. The covalently bound salt forming groups may be strongly acidic (e.g., sulfonic acid or sulfuric acid) or weakly acidic (e.g., carboxylic acid). In general, those types of ion-exchangers suitable for use in ion-exchange chromatography and for such applications as deionization of water are suitable for use in these controlled release drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975), incorporated by reference herein.

Resins suitable for use in the present invention include, but are not limited to Amberlite IRP-69 (Rohm and Haas) INDION 224, INDION 244, and INDION 254 (Ion Exchange (India) Ltd.). These resins are sulfonated polymers composed of polystyrene cross-linked with divinylbenzene. Any ion-exchange resins currently available and those that should become pharmaceutically acceptable and available in the future can also be used. Commercial sources of ion exchange resins that are either pharmaceutically acceptable or may become pharmaceutically acceptable in the future include, but are not limited to, Rohm and Haas, The Dow Chemical Company, and Ion Exchange (India) Ltd.

The size of the ion-exchange particles should be less than about 2 millimeter, more preferably below about 1000 micron, more preferably below about 500 micron, and most preferably below about 150 micron. Commercially available ion-exchange resins (Amberlite IRP-69, INDION 244 and INDION 254) have a particle size range less than 150 microns.

Drug is bound to the resin by exposure of the resin to the drug in solution via a batch or continuous process (such as in a chromatographic column). The drug-resin complex thus formed is collected by filtration and washed with an appropriate solvent to insure removal of any unbound drug or by-products. The complexes are usually air-dried in trays. Such processes are described in, for example, U.S. Pat. Nos. 4,221,778, 4,894,239, and 4,996,047.

Binding of drug to resin can be accomplished according to four general reactions. In the case of a basic drug, these are: (a) resin (Na-form) plus drug (salt form); (b) resin (Na-form) plus drug (as free base); (c) resin (H-form) plus drug (salt form); and (d) resin (H-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

B. Taste Masking Coatings

Milnacipran-containing resin particles can be coated with taste-masking coating. Taste-masking coating prevents the release of drug within the mouth and insures that no unpleasant, bitter taste is experienced by the patient consuming the dosage form.

The cationic polymer Eudragit® E 100 (Rohm Pharma) carries amino groups. Its films are, therefore, insoluble in the neutral medium of saliva, but dissolve by salt formation in the acid environment of the stomach. Such film coatings with a thickness of approximately 10 micrometers prevent medication with a bitter or revolting taste from dissolving in the mouth upon ingestion or during swallowing. The protective film dissolves quickly in the stomach allowing for the active ingredient to be released. A sugar coating may be used to accomplish similar taste-masking effect, albeit coating must be over 100 times thicker and these larger particles may result in tickling or irritating the throut.

C. Enteric Coatings

In some embodiments drug-resin complexes are coated with a pH sensitive polymer which is insoluble in the acid environment of the stomach, and soluble in the more basic environment of the GI tract. The outer coating is thus an enteric coating; such dosage form is designed to prevent drug release in the stomach. Preventing drug release in the stomach has the advantage of reducing side effects associated with irritation of the gastric mucosa. Avoiding release within the stomach can be achieved using enteric coatings known in the art. The enteric coated formulation remains intact or substantially intact in the stomach, however, once the formulation reaches the small intestines, the enteric coating dissolves and exposes either drug-containing ion-exchange resin particles or drug-containing ion-exchange resin particles coated with extended release coating.

The enteric coated particles can be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et. al., (Media, Pa.: Williams and Wilkins, 1995). Examples of suitable coating materials include but are not limited to cellulose polymers, such as cellulose acetate phthalate, hydroxypropyl cellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Rohm Pharma). Additionally the coating material may contain conventional carriers such as plasticizers, pigments, colorants, glidants, stabilization agents, and surfactants.

D. Extended Release Coatings

Extended release pharmaceutical compositions are obtained by complexing milnacipran with a pharmaceutically acceptable ion-exchange resin and coating such complexes with a substance that will act as a barrier to control the diffusion of the drug from its core complex into the gastrointestinal fluids.

Control of the release of drugs from drug-resin complexes is possible with the use of a diffusion barrier coating on the drug-resin complex particles. Several processing methods to achieve extended release coatings on drug loaded resin particles have been described (see, for example, U.S. Pat. Nos. 4,996,047, 4,221,778, and 4,894,239); any of these may be used to obtain the extended release milnacipran composition. Extended release coated milnacipran-resin complexes can also be prepared without the use of impregnating agents.

In general, any coating procedure which provides a contiguous coating on each particle of drug-resin complex without significant agglomeration of particles may be used. Coating procedures known in the pharmaceutical art including, but not limited to, fluid bed coating processes and microencapsulation may be used to obtain appropriate coatings. The coating materials may be any of a large number of natural or synthetic film-formers used singly, in admixture with each other, and in admixture with plasticizers (for example, Durkex 500 vegetable oil), pigments and other substances to alter the characteristics of the coating. In general, the major components of the coating should be insoluble in, and permeable to, water. However, it might be desirable to incorporate a water-soluble substance, such as methyl cellulose, to alter the permeability of the coating. The coating materials may be applied as a suspension in an aqueous fluid or as a solution in organic solvents. The water-permeable diffusion barrier may consist of ethyl cellulose, methyl cellulose and mixtures thereof. The water-permeable diffusion barrier may also consist of water insoluble synthetic polymers sold under the trade name Eudragit® (Rohm Pharma), such as Eudragit RS, Eudragit RL, Eudragit NE and mixtures thereof. Other examples of such coating materials can be found in the Handbook of Pharmaceutical Excipients, Ed. By A. Wade and P. J. Weller, (1994).

As used herein, the term water-permeable is used to indicate that the fluids of the alimentary canal will permeate or penetrate the coating film with or without dissolving the film or parts of the film. Depending on the permeability or solubility of the chosen coating (polymer or polymer mixture) a lighter or heavier application thereof is required to obtain the desired release rate.

U.S. Pat. No. 4,221,778 to Raghunathan describes the addition of solvating agents such as polyethylene glycol to the system in order to reduce the swelling of the drug-loaded resins and prevent the fracturing of the extended release coating. The solvating agent can be added as an ingredient in the resin drug complexation step or preferably, the particles can be treated with the solvating agent after complexing. This treatment has not only been found to help the particles retain their geometry, but has enabled the effective application of diffusion barrier coatings such as ethylcellulose to such particles. Other effective solvating (impregnating) agent candidates include, for example, propylene glycol, glycerin, mannitol, lactose and methylcellulose. Up to about 30 parts by weight (normally 10-25 parts) of the solvating agent to 100 parts by weight of the resin has been found to be effective. EP 171,528, EP 254,811, and EP 254,822 all disclose similar impregnation treatments in order to improve coatability of resin complexes.

Control of the release of drugs from drug-resin complexes has been achieved by the direct application of an ethylcellulose diffusion barrier coating to particles of such complexes in the absence of an impregnating agent, provided that the drug content of the complexes was above a critical value. U.S. Pat. No. 4,996,047, Kelleher et al., discloses extended release coated drug-resin complexes wherein the drug comprises more than about 38% by weight (for irregularly shaped particles) of the dry drug-resin complex (based on the free acid or base of drug). In order to achieve this relatively high loading, a method of complexing drug to resin is provided whereby the drug is combined in its basic form with the resin in its acidic form (or visa versa). Since no ionic by-products are formed in such a reaction, very high loading levels are achieved. A similar scheme was disclosed in U.S. Pat. No. 4,894,239 to Nonomura, et al, with the free form of the drug being formed as part of a continuous process. U.S. Pat. No. 4,894,239 states the drug-resin complex should contain at least 80% of the theoretical ion adsorption amount, and more preferably should contain about 85 to 100% of theoretical ion adsorption amount, to produce a stable coating on the final drug-resin complex.

U.S. Pat. No. 5,186,930, Kogan et al. discloses drug-resin particles coated with a first inner coating of wax and a second outer coating of a polymer to achieve extended release. The inner wax coating prevents the swelling of the resins and subsequent rupturing of the extended release polymer coating.

In addition to Inown methods of processing drug-loaded resins to obtain stable extended release coatings, it was found that coating of milnacipran loaded ion-exchange resins with an acrylic polymer based coating (Eudragit RS) results in a stable extended release composition without use of impregnating agents even when the drug loading is conducted by binding the salt form of the drug with the salt form of the resin, rather than binding the free base of the drug with resin in its acidic form as described by Kelleher et al and Nonomura et al. Milnacipran-resin complexes obtained by binding the salt form of the drug with the salt form of the resin have drug loadings lower than Kelleher et al and Nonomura et al reported as necessary to obtain stable extended release coatings without the use of impregnating agents.

E. Delayed Release Coatings

In some embodiments drug-resin complexes are coated with a pH sensitive polymer which is insoluble in the acid environment of the stomach, insoluble in the environment of the small intestines, and soluble in the conditions within the lower small intestine or upper large intestine (eg, above pH 7.0). Such a delayed release form is designed to prevent drug release in the upper part of the gastrointestinal (GI) tract.

The delayed release particles can be prepared by coating drug-containing microparticles with a selected coating material. Preferred coating materials are comprised of bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit.RTM. (Rohm Pharma; Westerstadt, Germany), including Eudragit.RTM. L30D-55 and L100-55 (soluble at pH 5.5 and above), Eudragit.RTM. L-100 (soluble at pH 6.0 and above), Eudragit.RTM. S (soluble at pH 7.0 and above, as a result of a higher degree of esterification), and Eudragits.RTM. NE, RL and RS (water-insoluble polymers having different degrees of permeability and expandability); vinyl polymers and copolymers such as polyvinyl pyrrolidone, vinyl acetate, vinyfacetate phthalate, vinylacetate crotonic acid copolymer, and ethylene-vinyl acetate copolymer; enzymatically degradable polymers such as azo polymers, pectin, chitosan, amylose and guar gum; and shellac. Combinations of different coating materials may also be used. Multi-layer coatings using different polymers may also be applied.

The preferred coating weights for particular coating materials may be readily determined by those skilled in the art by evaluating individual release profiles for drug loaded ion exchange resins with different quantities of various coating materials.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers are, but not limited to, polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Delayed release coated particles can be administered simultaneously with an immediate release dose of the drug. Such a combination produces the modified release profile referred to as “pulsatile release”. By “pulsatile” is meant that drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Optionally, a second pulse is followed by a second time interval (lag time) during which very little or no drug is released from the dosage form, after which a third dose is then released.

The first pulse of the pulsatile release composition can be obtained by administering unmodified drug, uncoated drug-resin particles, taste-masked coated drug-resin particles, or, in some cases, enteric coated drug-resin particles along with delayed release coated particles that provide a second pulse.

In some cases it may be advantageous to combine an immediately releasing dose of drug (eg, unmodified drug, uncoated drug-resin particles, or taste masking coated drug-resin particles) with enteric coated drug-resin particles to create a pulsatile profile. In this case the first pulse will occur substantially immediately and the second pulse will occur once the enteric coating has dissolved (in the upper small intestines).

In order to create a final dosage form with three pulses, an immediate release dose of drug (e.g., unmodified drug, uncoated drug-resin particles, or taste masking coated drug-resin particles) can be combined with enteric coated drug-resin particles and delayed release coated drug resin particles.

### II. Formulations Comprising Multiparticulate Milnacipran Compositions

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients.

A. Liquid Suspension

Typically, the carrier in a liquid formulation will include water and/or ethanol, flavorings (bubblegum is a favorite for pediatric use) and colorings (red, orange, and purple are popular).

The coated drug-resin particles are suitable for suspending in an essentially aqueous vehicle with the only restrictions on its composition being (i) an absence of, or very low levels of ionic ingredients, and (ii) a limitation on the concentrations of water-miscible organic solvents, such as alcohol, and the pH to those levels which do not cause dissolution of the diffusion barrier and enteric coatings. Liquid oral dosage forms include aqueous and nonaqueous solutions, emulsions, suspensions, and solutions and/or suspensions reconstituted from non-effervescent granules, containing suitable solvents, emulsifying agents, suspending agents, diluents, sweeteners, coloring agents, and flavoring agents. Preservatives may or may not be added to the liquid oral dosage forms. Specific examples of pharmaceutically acceptable carriers and excipients that may be used to formulate oral dosage forms are described in U.S. Pat. No. 3,903,297 to Robert.

In preparing the liquid oral dosage forms, the drug-resin complexes are incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. The most preferred carrier is a suspension of the pharmaceutical composition in an aqueous vehicle containing a suitable suspending agent. Suitable suspending agents include Avicel RC-591 (a microcrystalline cellulose/sodium carboxymethyl cellulose mixture available from FMC), guar gum and the like. Such suspending agents are well known to those skilled in the art.

Although water itself may make up the entire carrier, typical liquid formulations preferably contain a co-solvent, for example, propylene glycol, glycerin, sorbitol solution, to assist solubilization and incorporation of water-insoluble ingredients, such as flavoring oils and the like into the composition.

B. Chewable, Crushable, or Rapidly Dissolving Tablets

In some embodiments coated drug-resin complexes are incorporated into chewable tablets, crushable tablets, or tablets which dissolve rapidly within the mouth. Chewable tablet formulations containing coated particles are known in the pharmaceutical arts (see for instance the textbook “Pharmaceutical dosage form—tablets” Vol. 1 edited by H A Lieberman et al. Marcel Dekker, Inc. (1989). Crushable tablets are the conventional tablets that have the same in vitro and in vivo performance regardless of their physical integrity, i.e. tablets can be crushed and administered as a powder, e.g. on apple sauce or mixed with water and syringed into a nasogastric or jujunostomy tube. The crushable tablets can be prepared using methods of tablet manufacturing known in the pharmaceutical art. Fast dissolving tablets containing coated particles are described, for example, in U.S. Pat. No. 6,596,311.

C. Gels

In some embodiments coated drug-resin complexes are incorporated into gels. Ion-exchange resin containing gel compositions are known in the art, see, for example, U.S. Pat. No 4,837,255.

D. Reconstitutable Dosage Units

Coated drug-resin complexes can be formulated into a granular material and packaged in a sachet, capsule or other suitable packaging in unit dose. Such granular material can be reconstituted at the time of use into a suitable vehicle such as water. The granular material may contain excipients that facilitate the dispersion of the particles in water. Formulations of this type have been disclosed in U.S. Pat. No 6,077,532.

Other optional ingredients well known to the pharmaceutical art may also be included in amounts generally known for these ingredients, for example, natural or artificial sweeteners, flavoring agents, colorants and the like to provide a palatable and pleasant looking final product, antioxidants, for example, butylated hydroxy anisole or butylated hydroxy toluene, and preservatives, for example, methyl or propyl paraben or sodium benzoate, to prolong and enhance shelf life.

### III. Combinations with Other Active Compounds

Other drugs may be simultaneously administered in the same dosage form, or in separate dosage forms, and/or separately administered. Acidic or basic drugs may be administered either as complexes with ion-exchange resins or as unbound compounds. The drug-containing ion-exchange particles may be coated with a substance that will act as a barrier to control the diffusion of the drug from its core complex into the gastrointestinal fluids and/or optionally coated with a film of a polymer which is insoluble in the acid environment of the stomach, and soluble in the basic environment of lower GI tract.

The milnacipran can be administered adjunctively with other active compounds such as analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

Specific examples of compounds that can be adjunctively administered with milnacipran include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, dapoxetine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone. nadolol, naproxen, naratriptan, nefazodone, neurontin, nomifesivie, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phemnetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

By adjunctive administration is meant simultaneous administration of the compounds, in the same dosage form, simultaneous administration in separate dosage forms, and separate administration of the compounds.

### IV. Methods of Administration

The formulation can be administered to any patient in need thereof. Although preferred patients are human, typically any mammal including domestic animals such as dogs, cats and horses, may also be treated.

The amount of the active ingredients to be administered is chosen based on the amount which provides the desired dose to the patient in need of such treatment to alleviate symptoms or treat a condition.

Milnacipran has been used as an antidepressant in approximately 400,000 patients, and is known to be non-toxic in humans. Pharmacokinetic studies have shown that oral doses of milnacipran are rapidly absorbed and extensively distributed in the body within 1-2 hours. Maximum plasma levels are quickly reached, with a half-life in humans of approximately 8 hours. Metabolism in the liver leads to the formation of ten chemically identified metabolites, although these metabolites represent only about 10% of the concentration of the parent drug. In humans, 90% of the parent drug is eliminated unchanged via the kidneys. This pharmacokinetic profile gives milnacipran certain pharmacokinetic advantages, such as low inter-individual variation in plasma levels, low potential for drug interactions, and limited impact on hepatic cytochrome P-450 systems. These pharmacokinetic properties differentiate milnacipran from most other antidepressant drugs and contribute to the good safety profile of milnacipran (Puozzo C. et al., 1996, Int. Clin. Psychopharmacol., 11:15-27; Caccia S., 1998, Clin. Pharmacokinet., 34:281-302; Puozzo C. et al., 1998, Eur. J. Drug Metab. Pharmacokinet., 23:280-286).

Milnacipran can be administered for the treatment of depression, for fibromyalgia syndrome, clironic fatigue syndrome, pain, attention deficit/hyperactivity disorder, and visceral pain syndromes (VPS) such as irritable bowel syndrome (IBS), noncardiac chest pain (NCCP), functional dyspepsia, interstitial cystitis, essential vulvodynia, urethral syndrome, orchialgia, and affective disorders, including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, migraine headache, and tension headache.

Adverse reactions to the oral administration of milnacipran typically include at least one of the following: nausea, vomiting, headache, dyspepsia, abdominal pain, insomnia, tremulousness, anxiety, panic attack palpitations, urinary retention, orthostatic hypotension, diaphoresis, chest pain, rash, weight gain, back pain, constipation, vertigo, increased sweating, agitation, hot flushes, tremors, fatigue, somnolence, dysoria, nervousness, dry mouth, and irritability.

The vomiting reflex is triggered by stimulation of chemoreceptors in the upper GI tract and mechanoreceptors in the wall of the GI tract which are activated by both contraction and distension of the gut wall as well as by physical damage. A coordinating center in the central nervous system controls the emetic response. The center is located in the parvicellular reticular formation in the lateral medullary region of the brain. Afferent nerves to the vomiting center arise from the abdominal splanchic and vagal nerves, vestibule-labyrinthine receptors, the cerebral cortex and the cehmoreceptors trigger zone (CTZ). The CTZ lies adjacent in the area postrema and contains chemoreceptors that sample both blood and cerebro spinal fluid. Direct links exist between the emetic center and the CTZ. The CTZ is exposed to emetic stimuli of endogenous origin and to stimuli of exogenous origin such as drugs. The efferent branches of the cranial nerves V, VII, and IX, as well as the vagus nerve and sympathetic trunk produce the complex coordinated set of muscular contractions, cardiovascular responses and reverse peristalsis that characterizes vomiting. The area postrema is rich in dopamine receptors as well as 5-hydroxytryptamine (5HT) receptors.

In cases where the composition comprises an enteric coating, such compositions result in a release profile characterized by a 0.05-2 hours lag time period during which less than 20% of the total milnacipran dose is released into the stomach.

Exemplification

The present invention will be further understood by reference to the following non-limiting examples.

Analytical and Manufacturing Procedures

Uncoated drug-resin complexes were analyzed for drug content in the following manner: An accurately weighed sample (about 300 mg for uncoated complexes or 500 mg for coated complexes) was refluxed in a mixture of 10 mL DI water, 4.1 g of sodium acetate, and 85 mL of anhydrous ethanol for 3 hours. Following refluxing, the mixture was cooled, transferred into a 100 mL volumetric flask with the aid of DI water, and the volume was brought up to 100 mL with water. The resulting solution was analyzed for drug content via HPLC.

Determinations of drug release from drug-resin complexes were performed with a Distek Dissolution Apparatus equipped with paddles rotating at 125 rpm. In all instances the release medium was maintained at 37° C. Samples obtained at various timepoints were analyzed via HPLC.

Coating was carried out in a fluidized bed coating apparatus, GPCG-1 (Glatt Air Techniques, Inc.).

### EXAMPLE 1

### Preparation of Milnacipran Loaded Ion-exchange Resins

Lot 1:

A. Loading of Milnacipran (HCl Salt) to Amberlite IRP-69 (Na-Form):

| Ingredient | Quantity/Batch |
|---|---|
| Milnacipran HCl | 400.00 g |
| Amberlite IRP-69, Na+ form | 700.00 g |
| DI Water USP | qs |

Procedure:

Amberlite IRP-69 Resin was pre-washed three times with 4L DI water. Washing was conducted by mixing the resin-water slurry for 5 minutes, allowing the resins to settle for 30 minutes, and decanting the supernatant. Three liters (3L) of DI water was added to the pre-washed resin particles and kept under stirring using a Lightning Mixer with propeller blades at 300 rpm. Milnacipran HCl was added to the resin slurry while mixing. Mixing was continued for 2 hours. The supernatant from the resulting mixture was decanted off after allowing the resins to settle for 30 minutes. The drug-loaded resin particles were then washed twice with 4L of DI water; washing was conducted by mixing the water-resin slurry for 5 minutes, allowing the resins to settle for 30 minutes, and decanting the supernatant. The resulting drug-resin complex was dried in a forced draft oven at 45° C. until the loss on drying was less than 10% (as measured with a Mettler Toledo Moisture Analyzer at 110° C.).

The resulting milnacipran-resin complexes had the following properties:

| | | | Drug Load | Drug Load |
|---|---|---|---|---|
| | | Drug Load | (mg Milnacipran | (% of |
| | Loss on | (mg Milnacipran | base/mg | theoretical |
| Lot # | Drying | HCl/mg complex) | dry complex) | maximum load) |
| 1 | 4.4% | 0.378 | 0.344 | 67% |

B. Release of Milnacipran from Uncoated Complexes

Drug release was determined at 37° C. by adding 500 mg of uncoated drug-resin complex to 900 mL of 0.05 M Phosphate Buffer, pH 6.8 plus 0.1 M sodium chloride in a dissolution vessel equipped with paddles rotating at 125 rpm.

The following release data was obtained, demonstrating that uncoated complex does not have any extended release properties:

| Cumulative | Lot 1 (uncoated complex) |
|---|---|
| Time (hrs) | Cumulative mg released |
| 0.5 | 178.6 |
| 1 | 180.3 |
| 3 | 180.2 |
| 5 | 180.3 |
| 8 | 180.7 |

C. Loading of Milnacipran (Free Base) to Amberlite IRP-69 (H-Form):

Lot 2:

Briefly, the hydrogen form of Amberlite IRP-69 ion exchange resin was generated by percolating 4N HCl through a bed of resin in the sodium form in a glass column equipped with a fritted disk. Following percolation with acid, the resins were washed with water and finally, with isopropyl alcohol. Resins were dried to a constant weight.

| Ingredient | Quantity/Batch |
|---|---|
| Milnacipran base | 203.5 g |
| Amberlite IRP-69, H+ form | 200 g |
| DI Water USP | qs |

Procedure:

Resin was added to DI water gradually while stirring. Milnacipran base was added to the slurry and stirring was continued for approximately 24 hours. The resulting slurry was filtered and washed with isopropyl alcohol. The complex was dried at 45° C. in a forced draft oven.

The resulting milnacipran-resin complexes had the following properties:

| | | Drug Load | Drug Load |
|---|---|---|---|
| | Loss on | (mg Milnacipran HCl/ | (mg Milnacipran base/ |
| Lot # | Drying | mg complex) | mg dry complex) |
| 2 | 4.2% | 0.523 | 0.476 |

### EXAMPLE 2

### Extended Release Coated Complexes

A. Preparation of Extended Release Coated Complexes

Lot 3:

Coating Composition:

| Ingredient | Quantity/Batch |
|---|---|
| Eudragit RS 30 D (Rohm Pharma | 500 g |
| Polymers) | |
| Triethyl Citrate FCC | 30 g |
| Talc USP | 75 g |
| Sycopharm (Red 30 Iron Oxide) | 1 g |
| Sycopharm (Yellow 10 Iron Oxide) | 3 g |
| DI Water USP | 670 g |
| Total | 1279 g |

Coated drug-resin complexes were prepared by coating uncoated drug resin-complexes of Example 1 (Lot 1). A coating suspension was prepared by combining the ingredients in the table above. The suspension was filtered through a #100 mesh screen and kept under constant stirring during the coating procedure. Coating was carried out in a fluid bed coating apparatus equipped with a Wuster Column (GPCG-1, Glatt Air Techniques, Inc.). Following coating, the product was dried briefly in the fluidized bed (15 minutes at 30° C.). Finally, the coated particles were cured in a forced draft oven for 24 hours at 40° C. The conditions for the coating procedure were as follows:

Coating Parameters:

| Parameter | Value |
|---|---|
| Load of uncoated drug-resin complex | 800 g |
| Atomizing Air Pressure | 2.1 bar |
| Nozzle Size | 0.8 mm |
| Spray Rate | 4.5-7.0 g/min |
| Product Temperature | 22-26° C. |
| Theoretical Coating Weight Gain (based on total | 23.5% |
| solids sprayed) | |

B. Release of Milnacipran from Extended Release Coated Complexes

Drug release was determined at 37° C. by adding 500 mg of extended release coated drug-resin complex to 900 mL of 0.05 M Phosphate Buffer, pH 6.8 plus 0.1 M sodium chloride in a dissolution vessel equipped with paddles rotating at 125 rpm.

The following release data was obtained, demonstrating that the coating applied to the milnacipran-resin complexes is capable of controlling the release of drug:

| Cumulative | Lot 3 (extended release coated complex) |
|---|---|
| Time (hrs) | Cumulative mg released |
| 0.5 | 35.7 |
| 1 | 52.0 |
| 3 | 95.4 |
| 5 | 107.6 |
| 8 | 121.2 |
| 12 | 132.8 |
| 16 | 136.2 |

### EXAMPLE 3

### Delayed Release and Extended Release Coated Complexes

A. Preparation of Delayed Release and Extended Release Coated Complexes

Lot 4:

Coating Composition:

| Ingredient | Quantity/Batch |
|---|---|
| Eudragit L30-D-55 (Rohm Pharma Polymers) | 666.66 g |
| Triethyl Citrate FCC | 30 g |
| Talc USP | 100 g |
| Dow Corning 7-9245 30% Simethicone | 1 g |
| Emulsion, USP | |
| DI Water USP | 483.34 g |
| Total | 1280 g |

Delayed Release and extended release drug-resin complexes were prepared by coating extended release drug resin-complexes of Example 2 (Lot 3). A coating suspension was prepared by combining the ingredients in the table above. The suspension was filtered through a #100 mesh screen and kept under constant stirring during the coating procedure. Coating was carried out in a fluid bed coating apparatus equipped with a Wurster Column (GPCG-1, Glatt Air Techniques, Inc.). Following the coating procedure, the product coating was cured in a forced draft oven for 6 hours at 40° C. The conditions for the coating procedure were as follows:

Coating Parameters:

| Parameter | Value |
|---|---|
| Load of extended release coated drug-resin complex | 440 g |
| Atomizing Air Pressure | 2.0 bar |
| Nozzle Size | 0.8 mm |
| Spray Rate | 4.5-7.0 g/min |
| Product Temperature | 23-24° C. |
| Theoretical Coating Weight Gain (based on total | 71.0% |
| solids sprayed) | |

B. Release Milnacipran from Delayed Release and Extended Release Coated Complex

Drug release was determined at 37° C. by adding 500 mg of dual coated drug-resin complex to 750 mL of 0.1 N HCl plus 0.1 N sodium chloride and incubating for 2 hours. After 2 hours 250 mL of 0.20 M tribasic sodium phosphate plus 0.1 N sodium chloride that has been equilibrated to 37° C. was added to the vessel in order to change the pH to 6.8. Incubation was then continued for a total of 16 hours at pH 6.8.

The following release data demonstrates (1) that the outer enteric coating results in the release of less than 10% of the total drug load when incubated in 0.1N HCl for two hours, and, furthermore, (2) that the inner extended release coating controls the release of drug once the outer enteric coating is dissolved at pH 6.8.

| | Lot 4 (delayed release and extended |
|---|---|
| Cumulative | release coated complex) |
| Time (hrs) | Cumulative mg released |
| 0.1 N HCl | |
| 1 | 3.1 |
| 2 | 5.9 |
| pH 6.8 buffer | |
| 2.5 | 39.3 |
| 3 | 53.5 |
| 5 | 76.9 |
| 7 | 84.7 |
| 10 | 89.3 |
| 14 | 91.5 |
| 18 | 91.8 |

Modifications and variations of the compositions and methods of use thereof will be obvious to those skilled in the art and are intended to come within the scope of the following claims.

We claim:

1. A multiparticulate milnacipran composition for oral administration comprising particles consisting of milnacipran complexed with an ion-exchange resin.

2. The composition of claim 1 wherein the ion-exchange resin particles are less than about 2 millimeter in diameter.

3. The composition of claim 1 wherein the ion-exchange resin particles are less than about 500 microns in diameter.

4. The composition of claim 1 wherein the ion-exchange resin particles are less than about 150 microns in diameter.

5. The composition of claim 1 wherein the particles are taste-masked immediate release particles, prepared by coating drug-containing particles with a polymer that is insoluble in the neutral environment of saliva, but dissolves in the acid environment of the stomach.

6. The composition of claim 1 wherein the particles are enteric coated particles, prepared by coating drug-containing particles with a polymer that is insoluble in the acidic environment of the stomach but dissolves in the neutral environment of the small intestines.

7. The composition of claim 1 wherein the particles are extended release particles, prepared by coating drug-containing particles with a polymer that forms water insoluble but water permeable membrane.

8. The composition of claim 1 wherein the particles are enteric coated-extended release particles, prepared by coating extended release drug particles with an enteric coating.

9. The composition of claim 1 wherein the particles are taste-masked extended release particles, prepared by coating extended release drug particles with a polymer that is insoluble in the neutral environment of saliva, but dissolves in the acid environment of the stomach.

10. The composition of claim 1 wherein the particles are delayed release particles, prepared by coating drug-containing particles with a polymer that remains insoluble in the acidic environment of the stomach and the environment of the upper small intestines, but dissolves in the lower small intestines or upper large intestines.

11. The composition of claim 1 formulated into a dosage form selected from the group consisting of a gel, capsule, soft gelatin capsule, tablet, chewable tablet, crushable tablet, rapidly dissolving tablet, and unit of use sachet or capsule for reconstitution.

12. The composition of claim 1 formulated into a liquid or liquid suspension.

13. The composition of claim 1 providing pulsatile release of milnacipran.

14. The composition of claim 1 in a liquid dosage form that provides delayed or extended release of milnacipran to produce a therapeutic effect over approximately 24 hours when administered to a patient in need, with diminished incidence and reduced intensity relative to one or more immediate release milnacipran side effects.

15. The milnacipran composition of claim 14, wherein the side effect is nausea.

16. The milnacipran composition of claim 14, wherein the side effects are selected from the group consisting of vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

17. The milnacipran composition of claim 14 having a milnacipran release profile that is characterized by release of less than approximately 20% of the total dose over a period up to two hours, followed by a slow or extended drug release.

18. The milnacipran composition of claim 17 wherein the defined period of time is between approximately four and approximately twenty-four hours.

19. The composition of claim 1 further comprising one or more additional active ingredients.

20. The composition of claim 19 wherein the active ingredients are selected from the group consisting of analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics, and anti-narcoleptics.

21. The composition of claim 1 in a dosage form delivering a dosage equivalent of between 5 and 500 mg milnacipran.

22. The composition of claim 21 in a dosage form delivering a dosage equivalent of between 100 and 400 mg milnacipran.

23. The milnacipran composition of claim 1, wherein the milnacipran is in the form of a therapeutically equivalent dose of either dextrogyral or levrogyral enantiomers of the milnacipran.

24. The milnacipran composition of claim 1, wherein the milnacipran is in the form of a therapeutically equivalent dose of a mixture of milnacipran enantiomers.

25. The milnacipran composition of claim 1, wherein the milnacipran is in the form of a therapeutically equivalent dose of the active metabolite of milnacipran.

26. The milnacipran composition of claim 1, wherein the milnacipran is in the form of a therapeutically equivalent dose of para-hydroxy-milnacipran (F2782).

27. A method of treating a patient in need thereof comprising administering to the patient the composition of claim 1.

28. A method of making an oral formulation of milnacipran as defined by claim 1, comprising complexing milnacripran with ion-exchange resin particles and, optionally, coating the drug particles with one or more polymer layers.
